(19) United States
(12) Patent Application Publication
(10) Pub. No.: US 2009/0242805 A1
(43) Pub. Date: Oct. 1, 2009

(54) SYSTEMS AND METHODS FOR UNIFORM SEQUENTIAL LATERAL SOLIDIFICATION OF THIN FILMS USING HIGH FREQUENCY LASERS

(76) Inventor: James S. Im, New York, NY (US)

Correspondence Address: WilmerHale/Columbia University, 399 PARK AVENUE, NEW YORK, NY, 10022, US

(21) Appl. No.: 12/063,814

(22) PCT Filed: Aug. 16, 2006
(86) PCT No.: PCT/US2006/031929

§ 371 Date: Oct. 31, 2008

Related U.S. Application Data
(60) Provisional application No. 60/708,615, filed on Aug. 16, 2005.

(51) Int. Cl. A61N 5/00

(52) U.S. Cl. 250/492.2

(57) ABSTRACT

Under one aspect, a method for processing a thin film includes generating a first set of shaped beamlets from a first laser beam pulse, each of the beamlets of the first set of beamlets having a length defining the y-direction, a width defining the x-direction, and a fluence that is sufficient to substantially melt a film throughout its thickness in an irradiated film region and further being spaced in the x-direction from adjacent beamlets of the first set of beamlets by gaps; irradiating a first region of the film with the first set of shaped beamlets to form a first set of molten zones which laterally crystallize upon cooling to form a first set of crystallized regions including crystal grains that are substantially parallel to the x-direction and having a length and width substantially the same as the length and width of each of the shaped beamlets and being separated from adjacent crystallized regions by gaps substantially the same as the gaps separating the shaped beamlets; generating a second set of shaped beamlets from a second laser beam pulse, each beamlet of the second set of beamlets having a length, width, fluence, and spacing that is substantially the same as the length, width, fluence, and spacing of each beamlet of the first set of beamlets; and continuously scanning the film so as to irradiate a second region of the film with the second set of shaped beamlets to form a second set of molten zones that are displaced in the x-direction from the first set of crystallized regions, wherein at least one molten zone of the second set of molten zones partially overlaps at least one crystallized region of the first set of crystallized regions and crystallizes upon cooling to form elongations of crystals in said at least one crystallized region.

### CROSS-REFERENCE TO RELATED APPLICATIONS

This application claims the benefit of U.S. Provisional Patent Application No. 60/708,615, filed Aug. 16, 2005 and entitled “2-Shot SLS Scheme Optimization for High Frequency Lasers,” the entire contents of which are incorporated herein by reference.

### BACKGROUND

1. Field of the Invention

The disclosed subject matter generally relates to laser crystallization of thin films.

2. Related Art

In the field of semiconductor processing, a number of techniques have been described to convert thin amorphous silicon films into polycrystalline films. One such technique is sequential lateral solidification (“SLS”). SLS is a pulsed-laser crystallization process that can produce polycrystalline films having elongated crystal grains on substrates, such as, but not limited to, substrates that are intolerant to heat (e.g., glass and plastics). Examples of SLS systems and processes are described in commonly-owned U.S. Pat. Nos. 6,322,625, 6,368,945, 6,555,449, and 6,573,531, the entire contents of which are incorporated herein by reference.

SLS uses controlled laser pulses to melt a region of an amorphous or polycrystalline thin film on a substrate. The melted regions of film then laterally crystallize into a directionally solidified lateral columnar microstructure or a multitude of location-controlled large single-crystal regions. Generally, the melt/crystallization process is sequentially repeated over the surface of a thin film. One or more devices, such as image sensors and active-matrix liquid crystal displays (“AMLCD”) devices, can then be fabricated from the crystallized film. In the latter devices, a regular array of thin-film transistors (“TFTs”) is fabricated on a transparent substrate, and each transistor serves as a pixel controller.

When a polycrystalline material is used to fabricate devices having TFTs, the total resistance to carrier transport within the TFT channel is affected by the combination of barriers that a carrier has to cross as it travels under the influence of a given potential. Within a material processed by SLS, a carrier crosses many more grain boundaries if it travels perpendicularly to the long grain axes of the polycrystalline material, and thus experiences a higher resistance, than if it travels parallel to the long grain axes. Thus, in general, the performance of TFT devices fabricated on SLS-processed polycrystalline films depends on the microstructure of the film in the channel, relative to the film's long grain axes.

### SUMMARY OF THE INVENTION

Under one aspect, a method for processing a thin film includes generating a first set of shaped beamlets from a first laser beam pulse, each of the beamlets of the first set of beamlets having a length defining the y-direction, a width defining the x-direction, and a fluence that is sufficient to substantially melt a film throughout its thickness in an irradiated film region and further being spaced in the x-direction from adjacent beamlets of the first set of beamlets by gaps; irradiating a first region of the film with the first set of shaped beamlets to form a first set of molten zones which laterally crystallize upon cooling to form a first set of crystallized regions including crystal grains that are substantially parallel to the x-direction and having a length and width substantially the same as the length and width of each of the shaped beamlets and being separated from adjacent crystallized regions by gaps substantially the same as the gaps separating the shaped beamlets; generating a second set of shaped beamlets from a second laser beam pulse, each beamlet of the second set of beamlets having a length, width, fluence, and spacing that is substantially the same as the length, width, fluence, and spacing of each beamlet of the first set of beamlets; and continuously scanning the film so as to irradiate a second region of the film with the second set of shaped beamlets to form a second set of molten zones that are displaced in the x-direction from the first set of crystallized regions, wherein at least one molten zone of the second set of molten zones partially overlaps at least one crystallized region of the first set of crystallized regions and crystallizes upon cooling to form elongations of crystals in said at least one crystallized region.

One or more embodiments include one or more of the following features. The at least one molten zone of the second set of molten zones partially overlaps two adjacent crystallized regions of the first set of crystallized regions and crystallizes upon cooling to form elongations of crystals in said two adjacent crystallized regions. The overlapping area between said at least one molten zone of the second set of molten zones and said two adjacent crystallized regions of the first set of crystallized regions forms a contiguous area bounding a substantially uniform crystal microstructure having crystal grains substantially parallel to the x-direction. Shaping each beamlet of the first and second sets of shaped beamlets to include at least one tapered end. The tapered end includes a trapezoid. The tapered end includes a triangle. Shaping each beamlet of the first and second sets of shaped beamlets to have a width to length aspect ratio of between 1:5 and 1:5000. Shaping each beamlet of the first and second sets of shaped beamlets to have a width between about 4 and 10 μm. The gaps have a size that is less than the beamlet width. The gaps of the first and second sets of shaped beamlets have a width that is about one half or less of the width of the beamlets of the first and second sets of shaped beamlets. The at least one molten zone of the second set of molten zones overlaps said at least one crystallized region of the first set of crystallized regions by a distance that is greater than the lateral growth length and less than twice the lateral growth length of one or more crystals in said at least one crystallized region. The at least one molten zone of the second set of molten zones overlaps said at least one crystallized region of the first set of crystallized regions by a distance that is less than about 90% and more than about 10% of the lateral growth length of one or more crystals in said at least one crystallized region. The at least one molten zone of the second set of molten zones overlaps said at least one crystallized region of the first set of crystallized regions by about 50% of the lateral growth length of one or more crystals in said at least one crystallized region. The at least one molten zone of the second set of molten zones overlaps said at least one crystallized region of the first set of crystallized regions by an amount selected to provide a set of predetermined crystalline properties to at least the overlap region. The set of predetermined crystalline properties are suitable for a channel region of a pixel TFT. Any given irradiated region of the film is irradiated by two or fewer pulses. The gaps include uncrystallized film. Providing computer controls for coordinating steps (a), (b), (c), and (d). Generating said first and second sets of shaped beamlets includes transmitting said first and second laser pulses through a mask. The mask comprises a single row of slits that transmit the first and second laser pulses. Generating said first and second laser pulses at a frequency greater than about 1 kHz. Generating said first and second laser pulses at a frequency greater than about 6 kHz. The film comprises silicon. Generating a third set of shaped beamlets from a third laser beam pulse, each beamlet of the third set of beamlets having a length, width, fluence, and spacing that is substantially the same as the length, width, fluence, and spacing of each beamlet of the first and second sets of beamlets; and continuously scanning the film so as to irradiate a third region of the film with the third set of shaped beamlets to form a third set of molten zones that are displaced in the x-direction from the first and second sets of crystallized regions, wherein at least one molten zone of the third set of molten zones partially overlaps at least one crystallized region of the second set of crystallized regions and crystallizes upon cooling to form elongations of crystals in said at least one crystallized region of the second set of crystallized regions. At least one molten zone of the third set of molten zones also partially overlaps at least one crystallized region of the first set of crystallized regions and crystallizes upon cooling to form elongations of crystals in said at least one crystallized region of the first set of crystallized regions. No molten zone of the third set of molten zones partially overlaps at least one crystallized region of the first set of crystallized regions. Fabricating a thin film transistor within at least one crystallized region of the first or second sets of crystallized regions, wherein the thin film transistor is tilted at an angle relative to an orientation of crystal grains within said at least one crystallized region. The angle is about 1-20°. The angle is about 1-5°.

Under another aspect, a system for processing a film includes a laser source providing a sequence of laser beam pulses; laser optics that shape each laser beam pulse into a set of shaped beamlets, each of the beamlets having a length defining the y-direction, a width defining the x-direction, and a fluence that is sufficient to substantially melt a film throughout its thickness in an irradiated region and further being spaced in the x-direction from adjacent beamlets by gaps; a stage for supporting the film and capable of translation in at least the x-direction; and memory for storing a set of instructions. The instructions include generating a first set of shaped beamlets from a first laser beam pulse; irradiating a first region of the film with the first set of shaped beamlets to form a first set of molten zones which laterally crystallize upon cooling to form a first set of crystallized regions including crystal grains that are substantially parallel to the x-direction and having a length and width substantially the same as the length and width of each of the shaped beamlets and being separated from adjacent crystallized regions by gaps substantially the same as the gaps separating the shaped beamlets; generating a second set of shaped beamlets from a second laser beam pulse; and continuously scanning the film so as to irradiate a second region of the film with the second set of shaped beamlets to form a second set of molten zones that are displaced in the x-direction from the first set of crystallized regions, wherein at least one molten zone of the second set of molten zones partially overlaps at least one crystallized region of the first set of crystallized regions and crystallizes upon cooling to form elongations of crystals in said at least one crystallized region.

One or more embodiments include one or more of the following features. The memory further includes instructions for partially overlapping said at least one molten zone of the second set of molten zones with two adjacent crystallized regions of the first set of crystallized regions which crystallizes upon cooling to form elongations of crystals in said two adjacent crystallized regions. The memory further includes instructions for providing an overlapping area between said at least one molten zone of the second set of molten zones and said two adjacent crystallized regions of the first set of crystallized regions which forms a contiguous area bounding a substantially uniform crystal microstructure having crystal grains substantially parallel to the x-direction. The laser optics shape each beamlet to include at least one tapered end. The laser optics shape each beamlet such that the tapered end includes a trapezoid. The laser optics shape each beamlet such that the tapered end includes a triangle. The laser optics shape each beamlet to have a width to length aspect ratio of between 1:5 and 1:5000. The laser optics shape each beamlet to have a width between about 4 and 10 μm. The laser optics shape the set of beamlets to have gaps of a width that is less than the beamlet width. The laser optics shape the set of beamlets to have gaps of a width that is about one half or less of the width of the beamlets. The memory further includes instructions for overlapping said at least one molten zone of the second set of molten zones with said at least one crystallized region of the first set of crystallized regions by a distance that is greater than the lateral growth length and less than twice the lateral growth length of one or more crystals in said at least one crystallized region. The memory further includes instructions for overlapping said at least one molten zone of the second set of molten zones with said at least one crystallized region of the first set of crystallized regions by a distance that is less than about 90% and more than about 10% of the lateral growth length of one or more crystals in said at least one crystallized region. The memory further includes instructions for overlapping said at least one molten zone of the second set of molten zones with said at least one crystallized region of the first set of crystallized regions by about 50% of the lateral growth length of one or more crystals in said at least one crystallized region. The memory further includes instructions for overlapping said at least one molten zone of the second set of molten zones with said at least one crystallized region of the first set of crystallized regions by an amount selected to provide a set of predetermined crystalline properties to at least the overlap region. The set of predetermined crystalline properties are suitable for a channel region of a pixel TFT. The memory further includes instructions for translating the film in the x-direction after irradiating the first region of the film with the first set of shaped beamlets so as to irradiate the second region of the film with the second set of shaped beamlets. The laser optics include a mask. The mask includes a single row of slits. The laser source provides the sequence of laser pulses at a frequency greater than about 1 kHz. The laser source provides the sequence of laser pulses at a frequency greater than about 6 kHz. The film comprises silicon. The memory further includes instructions for generating a third set of shaped beamlets from a third laser beam pulse; and continuously scanning the film so as to irradiate a third region of the film with the third set of shaped beamlets to form a third set of molten zones that are displaced in the x-direction from the first and second sets of crystallized regions, wherein at least one molten zone of the third set of molten zones partially overlaps at least one crystallized region of the second set of crystallized regions and crystallizes upon cooling to form elongations of crystals in said at least one crystallized region of the second set of crystallized regions. The memory further includes instructions for partially overlapping said at least one molten zone of the third set of molten zones with at least one crystallized region of the first set of crystallized regions which crystallizes upon cooling to form elongations of crystals in said at least one crystallized region of the first set of crystallized regions. The memory further includes instructions for overlapping no molten zone of the third set of molten zones with at least one crystallized region of the first set of crystallized regions.

### BRIEF DESCRIPTION OF THE DRAWINGS

In the drawing,

FIG. 1 shows a diagram of a system for performing uniform SLS;

FIG. 2A shows a schematic of a mask for performing uniform SLS;

FIG. 2B is an illustration of a film irradiated by a laser beam shaped by the mask of FIG. 2A;

FIG. 3A shows a schematic of a mask for performing uniform SLS;

FIG. 3B is an illustration of a film irradiated by a laser beam shaped by the mask of FIG. 3A;

FIG. 4A shows a schematic of a mask for performing uniform SLS with a high frequency laser, according to certain embodiments;

FIG. 4B shows a schematic of the pattern of irradiation on a film from multiple laser beam pulses shaped by the mask of FIG. 4A according to certain embodiments;

FIG. 4C shows a schematic of the pattern of irradiation on a film from multiple laser beam pulses shaped by the mask of FIG. 4A according to certain embodiments; and

FIG. 4D is an illustration of a film irradiated by a laser beam shaped by the mask of FIG. 4A.

FIG. 5 is a schematic of the pattern of irradiation on a film from multiple laser beam pulses.

### DETAILED DESCRIPTION

The present application discloses systems and methods for using high-frequency pulsed lasers to perform uniform sequential lateral solidification of thin films, while reducing the number of edge areas that are present in regions where TFTs will be fabricated. The systems and methods provide crystallized areas with substantially uniform crystal orientation. SLS has been described using low frequency lasers, e.g., less than 1 kHz. Details of early SLS systems and methods may be found in U.S. Pat. No. 6,573,531, the entire contents of which are incorporated herein by reference. High frequency lasers may optionally be used in SLS processes, such as in the embodiments disclosed herein. High frequency lasers are readily available with substantially higher power than low frequency lasers (e.g., 1200 W at 6000 Hz vs. 500 W at 300 Hz), and can be used for other kinds of SLS processes such as line-scan SLS.

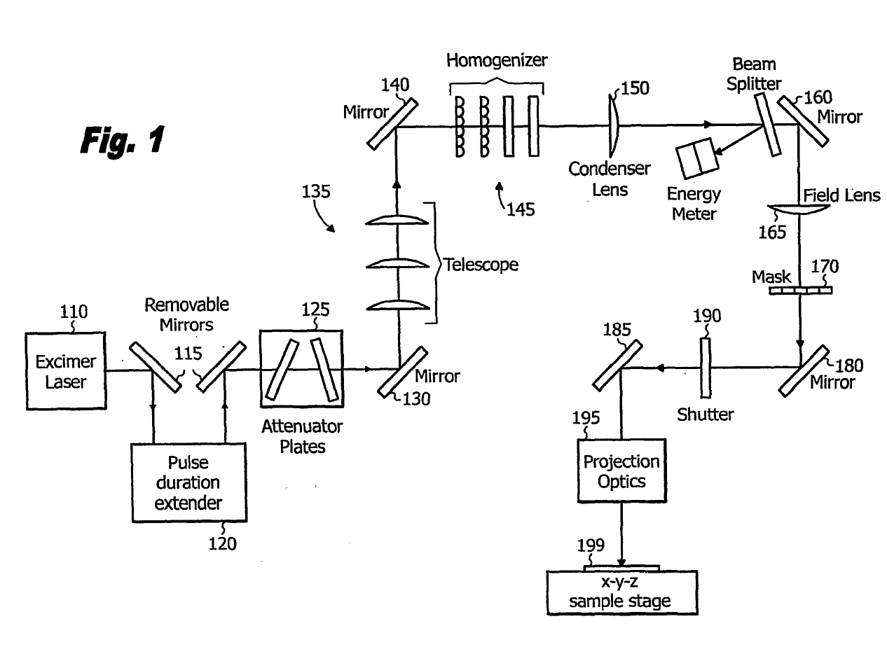

FIG. 1 shows an example of a system that can be used for SLS processes. A light source, e.g., an excimer laser 110 generates a pulsed laser beam which passes through a pulse duration extender 120 and attenuator plates 125 prior to passing through optical elements such as mirrors 130, 140, 160, telescope 135, homogenizer 145, beam splitter 155, and lens 165. The laser beam pulses then pass through a mask 170, which may be on a translation stage (not shown), and projection optics 195. The projection optics reduce the size of the laser beam and simultaneously increase the intensity of the optical energy striking substrate 199 at a desired location(s). The substrate 199 is provided on a precision x-y-z stage 200 that can accurately position the substrate 100 under the beam and assist in focusing or defocusing the image of the mask 170 produced by the laser beam at the desired location on the substrate.

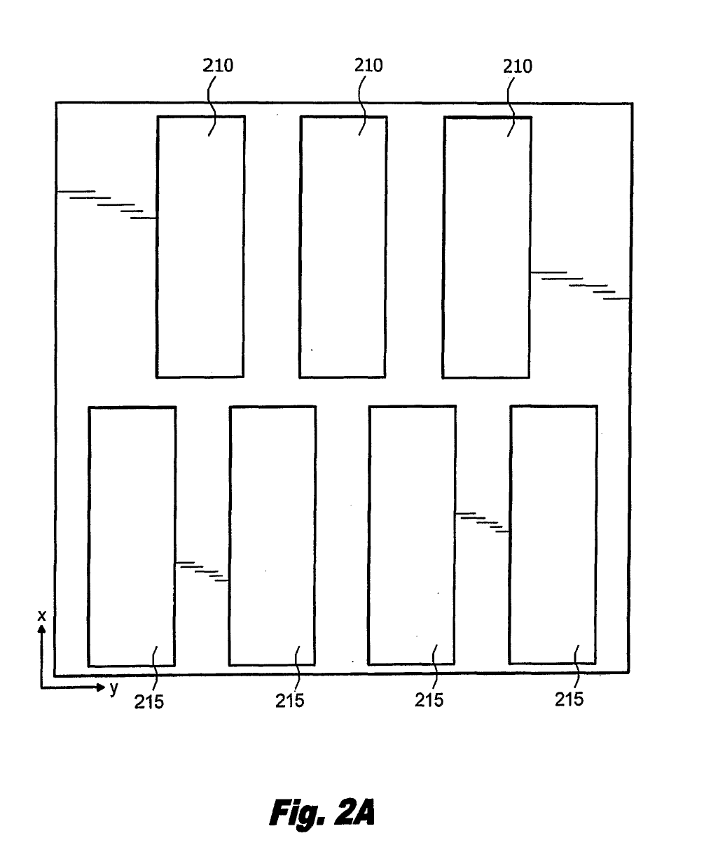

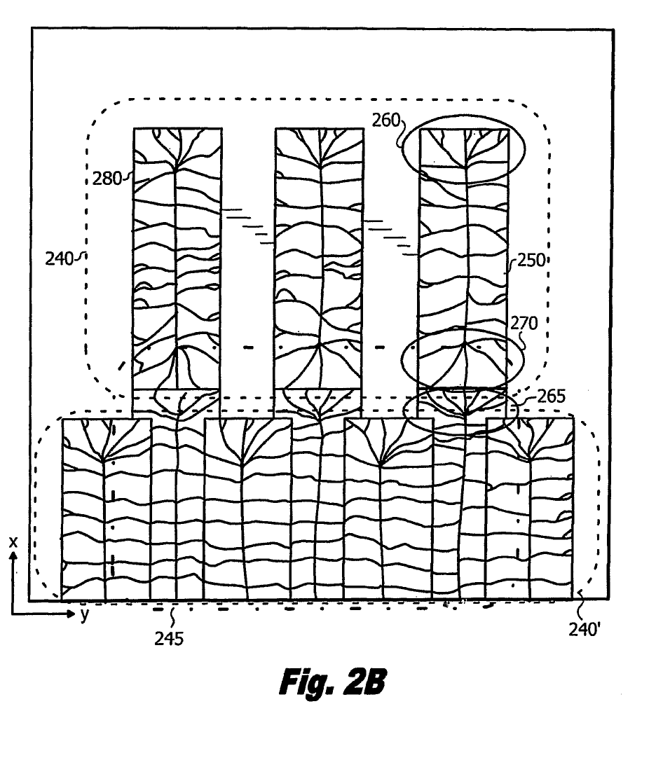

In one SLS scheme that leads to a crystalline film with a high level of uniformity, a given region of a thin film is irradiated with approximately two laser pulses, providing a relatively rapid way to produce polycrystalline semiconductor films. Further details of uniform grain structure SLS methods and systems may be found in PCT Publication No. WO 2002/086954, entitled “Method and System for Providing a Single-Scan, Continuous Motion Sequential Lateral Solidification,” the entire contents of which are incorporated herein by reference. FIG. 2 illustrates a mask such as that described in WO 2002/086954 that can be used in a uniform grain structure SLS scheme using the system of FIG. 1. The mask includes a plurality of rectangular slits 210, 215 which transmit and shape the laser beam to produce a plurality of beamlets that irradiate the thin film. The other (non-slit) portions of the mask are opaque. One set of slits 210 is offset in the x and y axes from a second set of slits 215. It should be understood that the mask illustration is intended to be schematic only, and that the dimensions and aspect ratios of the slits can vary greatly and are related to the desired speed of processing, the energy density needed to melt the film in an irradiated region, and the available energy per pulse. In general, the aspect ratio of width to length for a given slit can vary, e.g., between 1:5 and 1:200.

In operation, a stage moves the film continuously in the x direction, so that the long axes of the slits in the mask of FIG. 2A lie substantially parallel to the scan direction. As the film moves, the laser generates pulses at a given frequency, e.g., 300 Hz, which are shaped by the mask. The film velocity is selected so that as it moves, subsequent laser pulses irradiate overlapping regions of the film. Thus, as the film continuously advances, its whole surface is crystallized. FIG. 2B shows an exemplary illustration of a film that has been irradiated by two subsequent laser pulses. The film includes a first set of crystallized regions 245 that have been irradiated with a first pulse shaped by the mask of FIG. 2A into a first set of beamlets, and second and third sets of crystallized regions 240 and 240′ respectively that have been irradiated with a second pulse shaped by the mask of FIG. 2A. Specifically, the set of beamlets generated by slits 210 form second set of crystallized regions 240, and the set of beamlets generated by slits 215 form third set of crystallized regions 240′. When scanning the sample, the end portion crystal grains 270 of the second set of crystallized regions 240, generated by a second laser pulse, partially overlap the front portion crystal grains 265 of the first sets of crystallized regions 245, generated by a first laser pulse. The crystals of the third set of crystallized regions 240′, also generated by the second laser pulse, partially overlap the sides of the first set of crystallized regions 245, partially filling the space between the individual regions 280 of the first set of crystallized regions 245. As the film is scanned in the x direction, its entire surface can be crystallized.

Where a beamlet irradiates and thus melts an individual irradiated region 280 in a given row, upon cooling the crystals in that region grow from the edge of the region towards the middle of the region. Thus, in the central region 250 of the irradiated region, where the edges of the beamlet were aligned in the x direction (parallel to the scan), the crystal grains extend substantially in the y direction (perpendicular to the scan). Because the beamlets are relatively long, much of the crystallized area has crystal grains oriented in the y direction. In contrast, at the front and end regions 260 and 270 respectively, some of the crystals grow from the very ends of the region, so they extend substantially in the x direction (parallel to the scan), and others grow at an angle to the scan direction. These regions are known as “edge areas.” Here, artifacts may arise because the edge of the beam, which is reproduced in the molten portion, leads to lateral growth of grains extending in from the edges at angles that are skewed relative to the desired direction of the lateral growth.

As mentioned above, the performance of a TFT that is later fabricated on the film is related to the crystal orientation of that film relative to the TFT orientation, i.e., is related to the number of grain boundaries that electrons must cross in the channel region of the TFT. Thus, in general it is desirable that the crystal grains of the grown film all extend substantially in the same direction, e.g., in the y direction, so that devices that are later fabricated on the film will have comparable (and low) numbers of grain boundaries in the channel region. Because the front and end portion crystal grains 260 and 270 have crystal orientations that extend in directions other than the preferred direction, devices fabricated in those regions will suffer reduced performance.

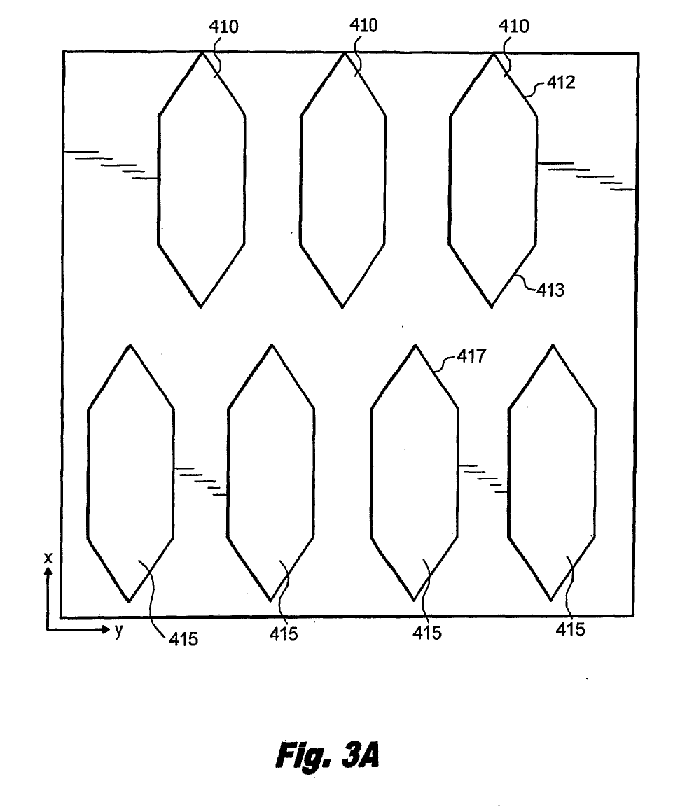

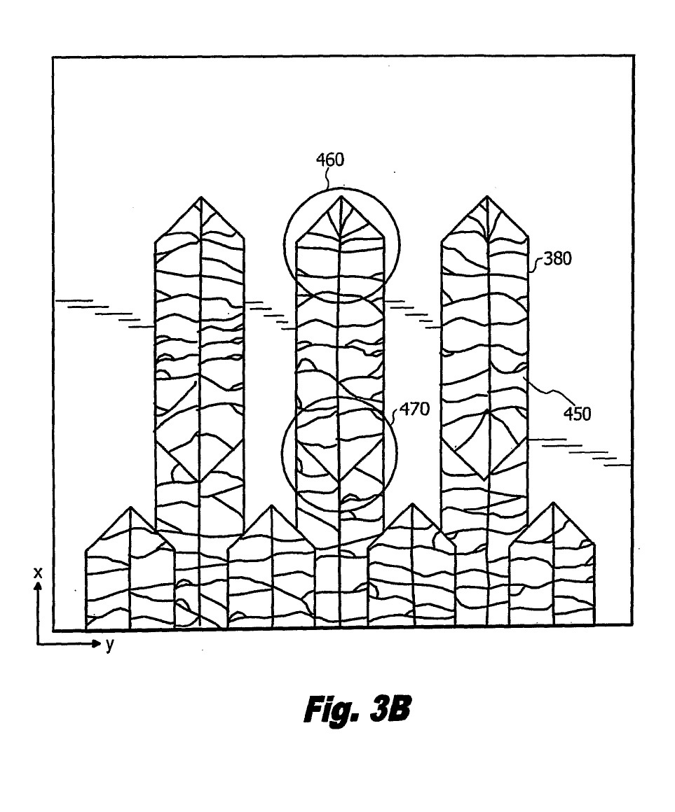

One way to address this issue is described in PCT Publication No. WO 2005/029546, entitled “Method and System for Providing a Continuous Motion Sequential Lateral Solidification For Reducing or Eliminating Artifacts, and a Mask for Facilitating Such Artifact Reduction/Elimination,” the entire contents of which are incorporated herein by reference. The mask may be modified by engineering tapered edges on the laser beamlets produced by the mask to ensure more parallel growth, as illustrated in FIG. 3A. Here, both ends 412 and 413 of each slit 410 in the mask have triangular-shaped sections that point away from the respective slit. As described above with respect to FIG. 2A, the slits transmit and thus shape the laser beam to provide a plurality of beamlets that irradiate the thin film. The other (non-slit) portions of the mask are opaque.

As described above for the case of rectangular beamlets, the sample moves continuously in the x direction. FIG. 3B shows an exemplary illustration of a film that has been irradiated multiple times with laser beamlets generated by the mask of FIG. 3A. Each individual irradiated region 380 includes central portion crystal grains 450 that extend substantially perpendicular to the scan direction (in the y direction), and front and end portion crystal grains 460 and 470 respectively, most of which extend substantially perpendicular to the scan direction, and a few of which extend substantially parallel to the scan direction. Here, because the ends of each beamlet are tapered, the crystal grains in the front and end portions of the irradiated region grow at an angle relative to the taper, yielding an orientation perpendicular to the scan direction. This can improve the alignment of the crystal grains in the “edge areas” relative to the remainder of the crystallized area.

When scanning the sample, end portion crystal grains 470 generated by a first pulse partially overlap with the front portion crystal grains 460 as well as the central portion crystal grains 450 generated by an earlier pulse. In this overlap region, the properly oriented grains 450 from the earlier pulse act as seed crystals for the end portion crystal grains from the second pulse, thus orienting the end portion crystal grains 470 in the desired y direction, substantially perpendicular to the scan direction.

Uniform grain structure SLS typically uses an excimer laser with relatively low repetition rate and a high energy per pulse (e.g., 100-500 W power, 100-300 Hz frequency, 0.5-2 J energy per pulse). Because the pulse energy is relatively high, the total beam area can be made relatively large, for example 15-50 mm². This way, a large surface area can be simultaneously processed, taking advantage of the high pulse energy. Additionally, it is desirable to reduce the stage scanning velocity so that it can be moved with higher accuracy, so the beam has a large aspect ratio, which spreads the energy over a longer beamlet, for example 1-2 mm on the short axis and 15-25 mm on the long axis.

Relatively high-frequency excimer lasers can also be used for uniform grain structure SLS schemes (e.g., 3-6 kHz). For the same overall beam power, the energy per pulse for a high frequency laser will be lower than that for a low frequency laser. Due to the decreased energy per pulse, the area thereof also needs to be reduced in order to maintain sufficiently high energy density for complete melting (e.g., 10-20 times smaller). For example, for a given power and stage velocity, if a 300 Hz laser has 1 J/pulse and is focused to a width of 1 mm, a 3 kHz laser will have only 100 mJ/pulse and will therefore need to be focused to a width of 100 μm. As a result, however, the relative fraction of ‘edge area’ will increase by a factor of ten. This may become problematic if many devices fall into these edge areas.

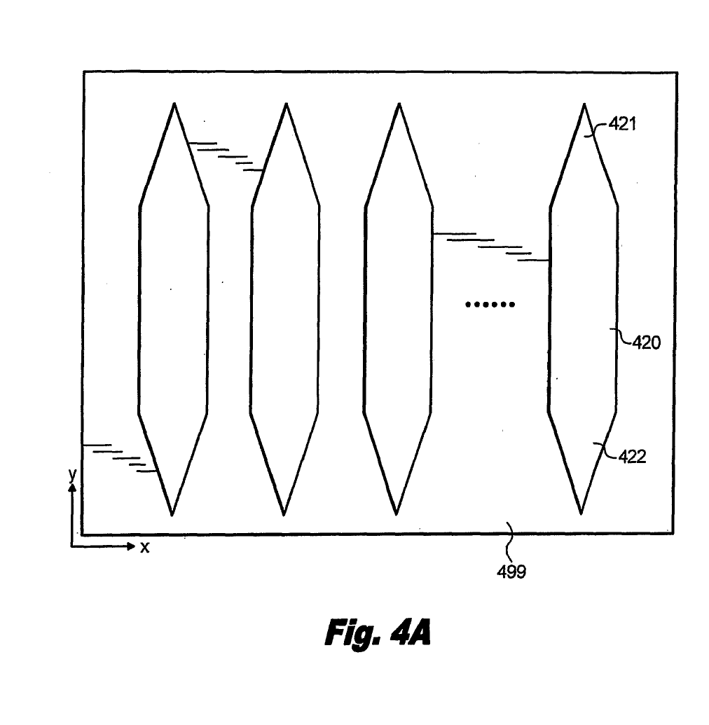

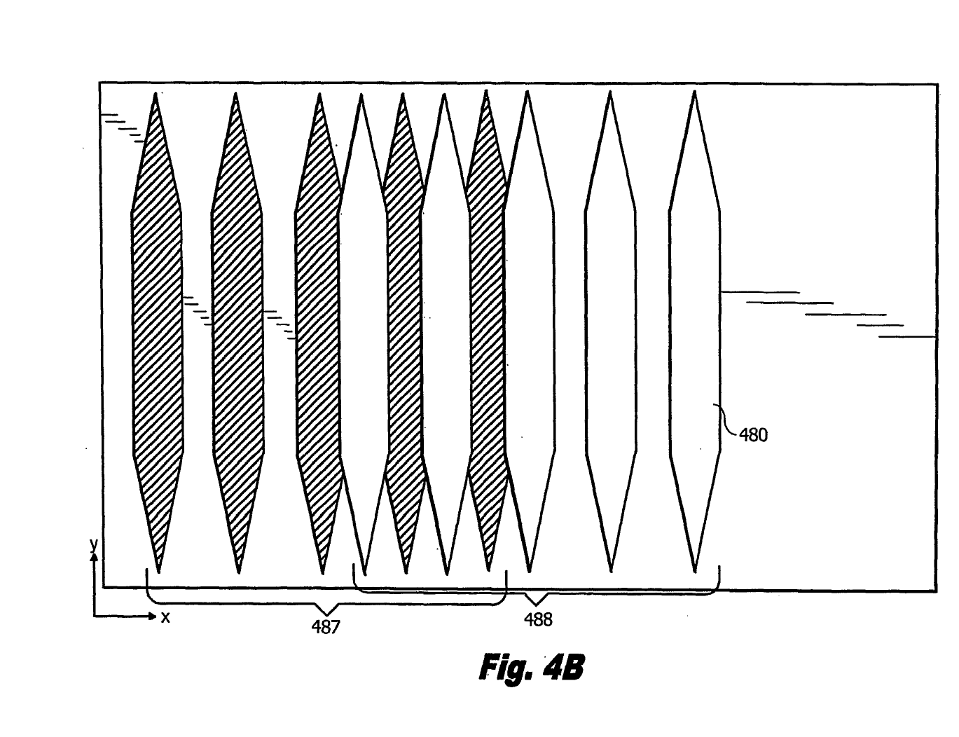

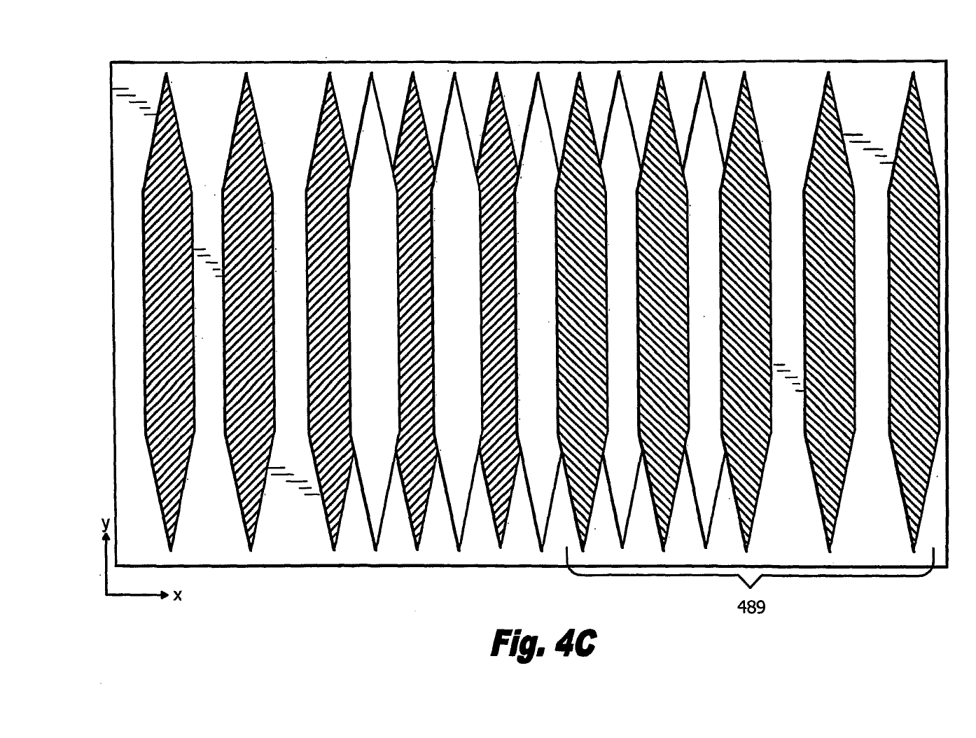

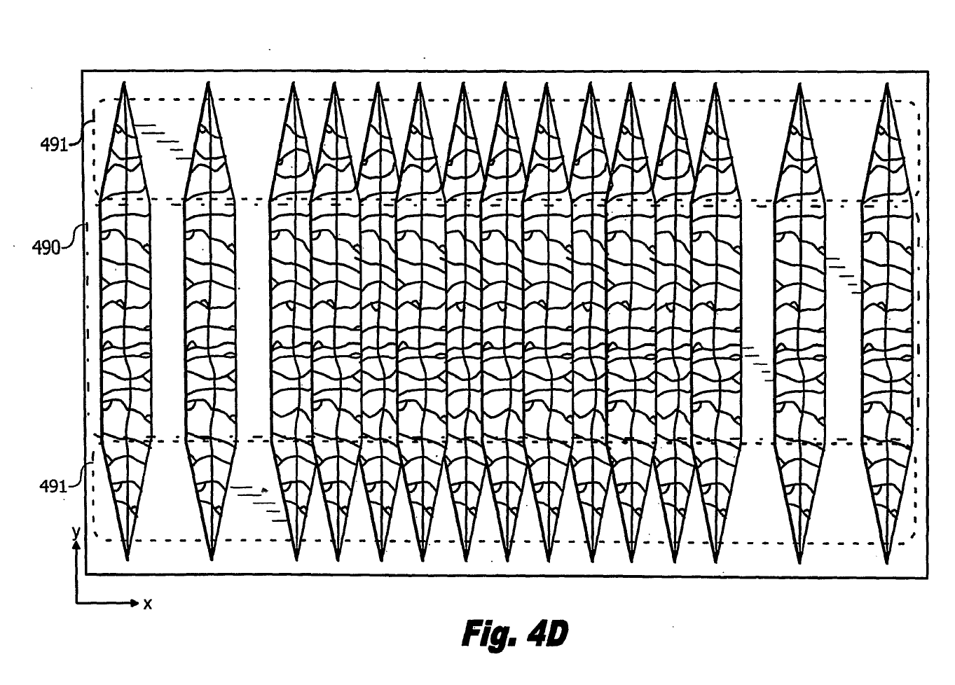

FIG. 4A illustrates an embodiment of a mask that can be used in the system of FIG. 1 to enable the use of high frequency lasers to perform uniform grain structure SLS. Mask 499 shapes a laser beam, generated by a high frequency laser (e.g., 3-6 kHz or higher) into a set of beamlets. Mask 499 includes a plurality of slits 420 that transmit the laser beam; the other (non-slit) portions of the mask are opaque and do not allow transmission of the laser beam. Each slit 420 has tapered ends 421 and 422 as described above regarding FIG. 3A and as further described in PCT Publication No. WO 2005/029546. The length of the slits 420 is oriented in the y-direction, and the width of the slits is oriented in the x-direction. As for the masks described above regarding FIGS. 2A and 3A, the length to width aspect ratio for the slits can vary, e.g., between 1:5 and 1:5000. Example beamlet widths at the sample can range between e.g., 4-10 μm. The gap between the slits is selected to be at least smaller than this value. For a more uniform material, it is selected to be significantly smaller as a larger overlap between the beams gives a more uniform grain width. For example, the gap may be between about 1-4 μm wide. In one example, the gap is about 1.5 μm wide and the slits are about 5.5 μm.

Although slits 4A are shown as having triangularly tapered edges, slits with other shapes can also be used. For example, slits with trapezoidal tapers and/or rounded edges may also be used. Rectangular slits may also be used. For further details on selecting beamlet and gap widths, as well as some other example slit shapes, see WO 2005/029546 and WO 2002/086954. Note also that while most embodiments have slits at a given spatial periodicity along the mask, in general not all of the dimensions and/or shapes for the slits and/or gaps need be identical.

In operation, a stage moves the film in the x-direction, so that the long axes of the beamlets lie substantially perpendicular to the direction of the scan. FIG. 4B shows a schematic of a film that has been irradiated by two subsequent laser pulses. The film includes a first set of crystallized regions 487 that has been irradiated with a first pulse shaped by the mask of FIG. 4A into a first set of beamlets, and a second set of crystallized regions 488 that has been irradiated with a second pulse shaped by the mask of FIG. 4A into a second set of beamlets. The first and second sets of crystallized regions 487, 488 are offset from each other in the x-direction by a distance that allows the second set to partially overlap the first set, e.g., by about 50%. Specifically, a subset of the individual irradiated regions 480 of the second set of crystallized regions 488 overlap a subset of the gaps between the individual irradiated regions 480 of the first set of crystallized regions 487. Another subset of the individual irradiated regions 480 of the second set of irradiated regions 488 extend beyond the first set of crystallized regions 487 in the x-direction. This subset includes gaps that have not yet been irradiated.

Microstructural details of the crystallized regions of the film have been omitted for clarity. However, it should be understood that the microstructure of the crystallized regions of the film are related to, among other things, the width and the energy density of the individual beamlets, the periodicity of the slits, and the overlap between adjacent irradiated regions. For example, in a first irradiated region, crystal growth typically begins at the edges of the irradiated region and grows inward. An example of this kind of growth can be seen, e.g., with regions 240 of FIG. 2B. Then, in an adjacent and overlapping second region, crystal growth begins from the overlapped existing crystal grains in the first region, generating elongated crystal grains. An example of this kind of growth can be seen, e.g., where the individual regions 280 within sets of regions 240′ and 245 overlap in FIG. 2B. In some embodiments, the second region may overlap the first region by a distance that is less than about 90% and more than about 10% of the lateral growth length of one or more crystals in the first region. The gap length is selected relative to the beamlet size to provide the desired overlap length, and thus to provide a set of predetermined crystalline properties to the crystallized region, including the overlap region. The set of predetermined crystalline properties may be suitable for later fabrication of devices in that region, e.g., a pixel TFT. In general, the relationship between processing parameters and the resulting film microstructure is well known in the art. Further details may be found in the patent references incorporated herein by reference.

FIG. 4C shows a schematic of the film of FIG. 4B after irradiation by a third laser pulse. The film now further includes a third set of crystallized regions 489 that has been irradiated with a third pulse shaped by the mask of FIG. 4A into a third set of beamlets. The third set of crystallized regions 489 partially overlaps the second set of crystallized regions 488, but not the first set of crystallized regions 487. Specifically, a subset of the individual irradiated regions 480 of the third set of crystallized regions overlap the unirradiated gaps between the individual irradiated regions 480 of the second set of crystallized regions, i.e., the gaps in subset of regions of the second set of crystallized regions 488 that extend beyond the first set of crystallized regions 487 in the x-direction. Note that in most embodiments, the displacement between the first and second irradiations is substantially the same as the displacement between the second and third irradiations, so assuming that the laser repetition rate is substantially constant, the film can be scanned at a substantially constant velocity. In summary, as the film is further scanned in the x-direction, the edges of the irradiated regions overlap with either the previously scanned region or will be overlapped by the following scanned region, thus uniformly crystallizing the film.

FIG. 4D shows an exemplary illustration of the microstructure of the film of FIG. 4C after irradiation with the three laser pulses. The film includes a central region 490 which is substantially uniformly crystallized, and “edge areas” 491 which are not uniformly crystallized and are not generally desirable for fabrication of TFTs, but which are spatially separated from the uniformly crystallized central region 490 and thus can readily be avoided or otherwise managed when fabricating the final device.

Although the drawings show only a single region 490 that has been uniformly crystallized using the exemplary methods and systems described herein, the disclosed methods and systems can be further applied to other regions of the same substrate, e.g., in overlapping regions above and/or below (e.g., in the +y or −y direction relative to) region 490. In such, the tapered ends formed in the subsequent region would be deliberately overlapped with the tapered ends of the previous region in the same way the ends are overlapped in FIG. 3B. While the crystal quality would not be perfectly uniform in this region, it would be satisfactory and could be avoided e.g., by methods described in greater detail below.

In most embodiments of the disclosed systems and methods, the relatively narrow individual irradiated regions substantially overlap with the narrow gaps between other irradiated regions, so that the gaps are substantially crystallized. If these gaps were not substantially crystallized, amorphous or polycrystalline film regions would remain in the gaps, and a device later fabricated on or partially overlapping the gap would not function properly. Most embodiments also provide a consistent amount of overlap between irradiated regions, so that the crystal quality of the film is consistent across the film's surface. In these, the position of the film relative to the laser beam is accurate to within some amount that provides satisfactory control of crystal growth. In some embodiments, the position of the film relative to the laser beam is accurate to within 0.5 μm, 0.2-0.3 μm, or even 0.1 μm. In one example, computer control (not shown) coordinates the film motion with the firing of the laser, thus providing relatively accurate film positioning relative to irradiation by the laser beam. This coordination is described in U.S. Patent Publication no. 2006/0102901, the entire contents of which are incorporated herein by reference. The frequency of the laser need not be precisely fixed; instead, the stage provides feedback regarding the film position to the computer control, so that when the film is in the correct position to irradiate with a laser pulse, the control instructs the laser to fire that pulse. Processing conditions, such as beam size, laser frequency, and stage velocity, may also improve the accuracy of the film position. Currently, the stage position relative to the laser beam can be controlled within about 0.5 μm, and with improvement of technology and experimental conditions, achieving 0.1 μm or better should be possible.

In the schemes illustrated in FIGS. 2A-2B and 3A-3B, some regions are irradiated by two pulses, but other regions are irradiated by more than two pulses. For example, in FIG. 2B regions 265 and 270 overlap, meaning two pulses have irradiated the overlap region. Then, when a next pulse irradiates the gap between the overlap region and the overlap region below it (in the −y direction), both overlap regions will be irradiated again by that next pulse. This means a total of three pulses irradiate a portion of the overlap regions; two pulses irradiate the remainder of the overlap regions; and one pulse irradiates the central portion of each irradiated area 280. In general, depending on the amount of overlap between the irradiated regions in the x and y directions, many pulses may irradiate a given region, while other regions are irradiated with few or even one pulse. The more pulses irradiate a region, the surface of the film physically changes. For example, as a film with an initially smooth surface is crystallized, there is mass flow which causes undulations in the film surface that follow the film microstructure. Where there are many irradiation pulses, the surface roughness will be worse than in regions where there are fewer irradiation pulses.

In most embodiments, non-uniformities at edge areas appear at the top and the bottom of each scanned area. Thus, relatively large regions of the film are free of edge areas and can be utilized for fabrication of TFTs of substantially uniform quality. The periodicity of the edge areas is not related to the dimension of the short axis of the beam. As noted above, in most embodiments, the short axis of the beam is significantly smaller than the long axis of the beam, so as to reduce the stage scanning velocity so that it can be moved with higher accuracy, and to also take advantage of the high pulse energy.

In some embodiments, when an array of TFTs is later fabricated on the film, the panel can be slightly tilted relative to the array orientation, so that the “edge areas” will not be collinear with the array, and thus not readily visible by eye. Instead, the edge areas may run through some devices but not its neighbors, so that the effect to the eye will be much less. In one or more embodiments, a small tilt angle such as 1-20°, or 1-5°, is used. U.S. Patent Publication No. 2005/0034653, entitled “Polycrystalline TFT Uniformity through Microstructure Misalignment,” the entire contents of which are incorporated herein by reference, provides some examples of locating TFTs on a silicon substrate relative to the long dimension grain boundaries of a uniformly crystallized film.

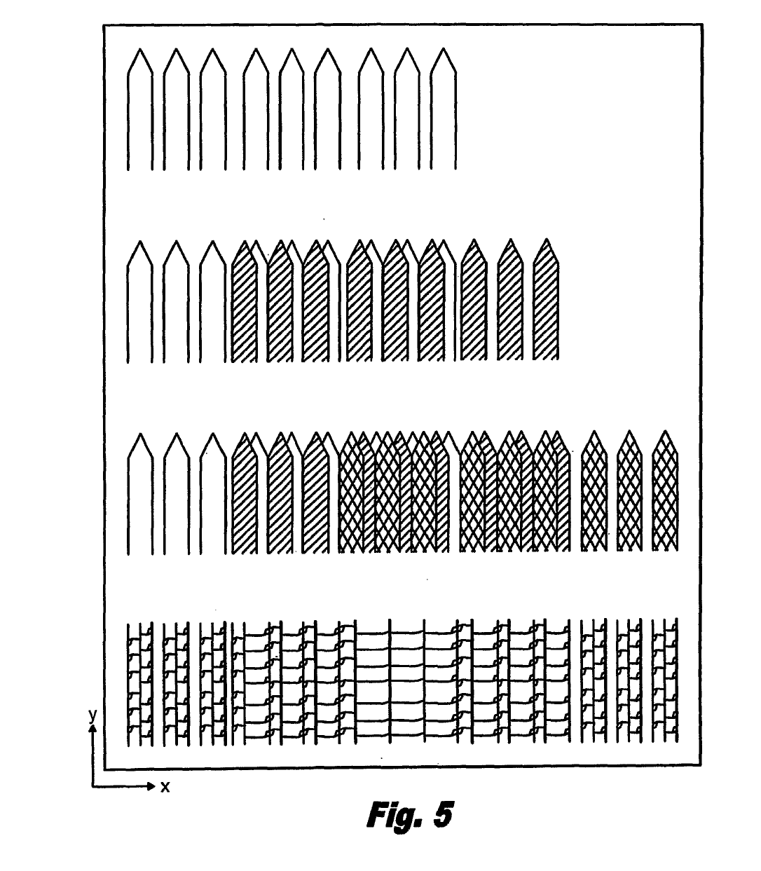

Although the embodiments described above are generally described with reference to irradiating a given area of the film with at most two laser pulses, i.e., “2-shot” SLS, it will be readily appreciated that other embodiments provide systems and methods for “n-shot” SLS, wherein a given region of film is irradiated with “n” laser pulses, e.g., 3, 4, or more. In some embodiments, the width, shape, periodicity, and number of slits and/or gaps in the mask, as well as the amount of displacement in the x-direction between each irradiation, are selected to provide the desired crystal structure with the desired number of laser pulses. In some embodiments, a second shaped laser pulse need not completely overlap a gap between crystallized regions generated by a first shaped pulse, but instead may partially overlap a crystallized region and partially overlap the gap adjacent that crystallized region. Then, a subsequent shaped laser pulse may irradiate either a portion or the remainder of gap, while also overlapping crystallized regions formed by the first and second shaped laser pulses. FIG. 5 illustrates an exemplary irradiation sequence wherein three laser pulses are used to generate an elongated crystal structure.

Other embodiments are within the following claims.

What is claimed is:

1. A method for processing a thin film, comprising:
(a) generating a first set of shaped beamlets from a first laser beam pulse, each of the beamlets of the first set of beamlets having a length defining the y-direction, a width defining the x-direction, and a fluence that is sufficient to substantially melt a film throughout its thickness in an irradiated film region and further being spaced in the x-direction from adjacent beamlets of the first set of beamlets by gaps;
(b) irradiating a first region of the film with the first set of shaped beamlets to form a first set of molten zones which laterally crystallize upon cooling to form a first set of crystallized regions including crystal grains that are substantially parallel to the x-direction and having a length and width substantially the same as the length and width of each of the shaped beamlets and being separated from adjacent crystallized regions by gaps substantially the same as the gaps separating the shaped beamlets;
(c) generating a second set of shaped beamlets from a second laser beam pulse, each beamlet of the second set of beamlets having a length, width, fluence, and spacing that is substantially the same as the length, width, fluence, and spacing of each beamlet of the first set of beamlets;
(d) continuously scanning the film so as to irradiate a second region of the film with the second set of shaped beamlets to form a second set of molten zones that are displaced in the x-direction from the first set of crystallized regions, wherein at least one molten zone of the second set of molten zones partially overlaps at least one crystallized region of the first set of crystallized regions and crystallizes upon cooling to form elongations of crystals in said at least one crystallized region.

2. The method of claim 1, wherein said at least one molten zone of the second set of molten zones partially overlaps two adjacent crystallized regions of the first set of crystallized regions and crystallizes upon cooling to form elongations of crystals in said two adjacent crystallized regions.

3. The method of claim 2, wherein the overlapping area between said at least one molten zone of the second set of molten zones and said two adjacent crystallized regions of the first set of crystallized regions forms a contiguous area bounding a substantially uniform crystal microstructure having crystal grains substantially parallel to the x-direction.

4. The method of claim 1, further comprising shaping each beamlet of the first and second sets of shaped beamlets to include at least one tapered end.

5. The method of claim 4, wherein the tapered end includes a trapezoid.

6. The method of claim 4, wherein the tapered end includes a triangle.

7. The method of claim 1, further comprising shaping each beamlet of the first and second sets of shaped beamlets to have a width to length aspect ratio of between 1:5 and 1:5000.

8. The method of claim 1, further comprising shaping each beamlet of the first and second sets of shaped beamlets to have a width between about 4 and 10 μm.

9. The method of claim 1, wherein the gaps have a size that is less than the beamlet width.

10. The method of claim 1, wherein the gaps of the first and second sets of shaped beamlets have a width that is about one half or less of the width of the beamlets of the first and second sets of shaped beamlets.

11. The method of claim 1, wherein said at least one molten zone of the second set of molten zones overlaps said at least one crystallized region of the first set of crystallized regions by a distance that is greater than the lateral growth length and less than twice the lateral growth length of one or more crystals in said at least one crystallized region.

12. The method of claim 1, wherein said at least one molten zone of the second set of molten zones overlaps said at least one crystallized region of the first set of crystallized regions by a distance that is less than about 90% and more than about 10% of the lateral growth length of one or more crystals in said at least one crystallized region.

13. The method of claim 1, wherein said at least one molten zone of the second set of molten zones overlaps said at least one crystallized region of the first set of crystallized regions by about 50% of the lateral growth length of one or more crystals in said at least one crystallized region.

14. The method of claim 1, wherein said at least one molten zone of the second set of molten zones overlaps said at least one crystallized region of the first set of crystallized regions by an amount selected to provide a set of predetermined crystalline properties to at least the overlap region.

15. The method of claim 14, wherein the set of predetermined crystalline properties are suitable for a channel region of a pixel TFT.

16. The method of claim 1, wherein any given irradiated region of the film is irradiated by two or fewer pulses.

17. The method of claim 1, wherein the gaps comprise uncrystallized film.

18. The method of claim 1, further comprising providing computer controls for coordinating steps (a), (b), (c), and (d).

19. The method of claim 1, wherein generating said first and second sets of shaped beamlets comprises transmitting said first and second laser pulses through a mask.

20. The method of claim 19, wherein said mask comprises a single row of slits that transmit the first and second laser pulses.

21. The method of claim 1, comprising generating said first and second laser pulses at a frequency greater than about 1 kHz.

22. The method of claim 1, comprising generating said first and second laser pulses at a frequency greater than about 6 kHz.

23. The method of claim 1, wherein the film comprises silicon.

24. The method of claim 1, further comprising:
generating a third set of shaped beamlets from a third laser beam pulse, each beamlet of the third set of beamlets having a length, width, fluence, and spacing that is substantially the same as the length, width, fluence, and spacing of each beamlet of the first and second sets of beamlets; and
continuously scanning the film so as to irradiate a third region of the film with the third set of shaped beamlets to form a third set of molten zones that are displaced in the x-direction from the first and second sets of crystallized regions, wherein at least one molten zone of the third set of molten zones partially overlaps at least one crystallized region of the second set of crystallized regions and crystallizes upon cooling to form elongations of crystals in said at least one crystallized region of the second set of crystallized regions.

25. The method of claim 24, wherein said at least one molten zone of the third set of molten zones also partially overlaps at least one crystallized region of the first set of crystallized regions and crystallizes upon cooling to form elongations of crystals in said at least one crystallized region of the first set of crystallized regions.

26. The method of claim 24, wherein no molten zone of the third set of molten zones partially overlaps at least one crystallized region of the of the first set of crystallized regions.

27. The method of claim 1, further comprising fabricating a thin film transistors within at least one crystallized region of the first or second sets of crystallized regions, wherein the thin film transistor is tilted at an angle relative to an orientation of crystal grains within said at least one crystallized region.

28. The method of claim 27, wherein the angle is about 1-20°.

29. The method of claim 27, wherein the angle is about 1-5°.

30. A system for processing a film, the system comprising:
a laser source providing a sequence of laser beam pulses;
laser optics that shape each laser beam pulse into a set of shaped beamlets, each of the beamlets having a length defining the y-direction, a width defining the x-direction, and a fluence that is sufficient to substantially melt a film throughout its thickness in an irradiated region and further being spaced in the x-direction from adjacent beamlets by gaps;
a stage for supporting the film and capable of translation in at least the x-direction;
memory for storing a set of instructions, the instructions comprising:
(a) generating a first set of shaped beamlets from a first laser beam pulse;
(b) irradiating a first region of the film with the first set of shaped beamlets to form a first set of molten zones which laterally crystallize upon cooling to form a first set of crystallized regions including crystal grains that are substantially parallel to the x-direction and having a length and width substantially the same as the length and width of each of the shaped beamlets and being separated from adjacent crystallized regions by gaps substantially the same as the gaps separating the shaped beamlets;
(c) generating a second set of shaped beamlets from a second laser beam pulse; and
(d) continuously scanning the film so as to irradiate a second region of the film with the second set of shaped beamlets to form a second set of molten zones that are displaced in the x-direction from the first set of crystallized regions, wherein at least one molten zone of the second set of molten zones partially overlaps at least one crystallized region of the first set of crystallized regions and crystallizes upon cooling to form elongations of crystals in said at least one crystallized region.

31. The system of claim 30, wherein the memory further includes instructions for partially overlapping said at least one molten zone of the second set of molten zones with two adjacent crystallized regions of the first set of crystallized regions which crystallizes upon cooling to form elongations of crystals in said two adjacent crystallized regions.

32. The system of claim 31, wherein the memory further includes instructions for providing an overlapping area between said at least one molten zone of the second set of molten zones and said two adjacent crystallized regions of the first set of crystallized regions which forms a contiguous area bounding a substantially uniform crystal microstructure having crystal grains substantially parallel to the x-direction.

33. The system of claim 31, wherein the laser optics shape each beamlet to include at least one tapered end.

34. The system of claim 33, wherein the laser optics shape each beamlet such that the tapered end includes a trapezoid.

35. The system of claim 33, wherein the laser optics shape each beamlet such that the tapered end includes a triangle.

36. The system of claim 30, wherein the laser optics shape each beamlet to have a width to length aspect ratio of between 1:5 and 1:5000.

37. The system of claim 30, wherein the laser optics shape each beamlet to have a width between about 4 and 10 μm.

38. The system of claim 30, wherein the laser optics shape the set of beamlets to have gaps of a width that is less than the beamlet width.

39. The system of claim 30, wherein laser optics shape the set of beamlets to have gaps of a width that is about one half or less of the width of the beamlets.

40. The system of claim 30, wherein the memory further includes instructions for overlapping said at least one molten zone of the second set of molten zones with said at least one crystallized region of the first set of crystallized regions by a distance that is greater than the lateral growth length and less than twice the lateral growth length of one or more crystals in said at least one crystallized region.

41. The system of claim 30, wherein the memory further includes instructions for overlapping said at least one molten zone of the second set of molten zones with said at least one crystallized region of the first set of crystallized regions by a distance that is less than about 90% and more than about 10% of the lateral growth length of one or more crystals in said at least one crystallized region.

42. The system of claim 30, wherein the memory further includes instructions for overlapping said at least one molten zone of the second set of molten zones with said at least one crystallized region of the first set of crystallized regions by about 50% of the lateral growth length of one or more crystals in said at least one crystallized region.

43. The system of claim 30, wherein the memory further includes instructions for overlapping said at least one molten zone of the second set of molten zones with said at least one crystallized region of the first set of crystallized regions by an amount selected to provide a set of predetermined crystalline properties to at least the overlap region.

44. The system of claim 43, wherein the set of predetermined crystalline properties are suitable for a channel region of a pixel TFT.

45. The system of claim 30, wherein the memory further includes instructions for translating the film in the x-direction after irradiating the first region of the film with the first set of shaped beamlets so as to irradiate the second region of the film with the second set of shaped beamlets.

46. The system of claim 30, wherein the laser optics comprise a mask.

47. The system of claim 46, wherein the mask comprises a single row of slits.

48. The system of claim 30, wherein the laser source provides the sequence of laser pulses at a frequency greater than about 1 kHz.

49. The system of claim 30, wherein the laser source provides the sequence of laser pulses at a frequency greater than about 6 kHz.

50. The system of claim 30, wherein the film comprises silicon.

51. The system of claim 30, wherein the memory further includes instructions for:
generating a third set of shaped beamlets from a third laser beam pulse; and
continuously scanning the film so as to irradiate a third region of the film with the third set of shaped beamlets to form a third set of molten zones that are displaced in the x-direction from the first and second sets of crystallized regions, wherein at least one molten zone of the third set of molten zones partially overlap partially overlaps at least one crystallized region of the second set of crystallized regions and crystallizes upon cooling to form elongations of crystals in said at least one crystallized region of the second set of crystallized regions.

52. The system of claim 51, wherein the memory further includes instructions for partially overlapping said at least one molten zone of the third set of molten zones with at least one crystallized region of the first set of crystallized regions which crystallizes upon cooling to form elongations of crystals in said at least one crystallized region of the first set of crystallized regions.

53. The system of claim 51, wherein the memory further includes instructions for overlapping no molten zone of the third set of molten zones with at least one crystallized region of the of the first set of crystallized regions.
